(19) United States
(12) Patent Application Publication
(10) Pub. No.: US 2016/0022610 A1
(43) Pub. Date: Jan. 28, 2016

(54) COMBINATION TREATMENT COMPRISING BUPROPION ( (±)-2-(TERT-BUTYLAMINO)-1-(3-CHLOROPHENYL)PROPAN-1-ONE) AND A SYMPATHOMIMETIC DRUG FOR WEIGHT CONTROL AND TREATING OBESITY

(71) Applicant: Thomas R. Winston, Leawood, KS (US)

(72) Inventor: Thomas R. Winston, Leawood, KS (US)

(21) Appl. No.: 14/337,661

(22) Filed: Jul. 22, 2014

(51) Int. Cl. A61K 31/137; A61K 45/06

(52) U.S. Cl. CPC A61K 31/137; A61K 45/06

(57) ABSTRACT

Compositions and methods for controlling weight gain and treating obesity are disclosed. Compositions include bupropion and a sympathomimetic drug. Methods for controlling weight include administering a composition including a combination of bupropion and a sympathomimetic drug. Methods for treating obesity include administering a composition including a combination of bupropion and a sympathomimetic drug. The active agents of the composition may be administered at a lower dosages than either drug separately with less side effect risk and allow for longer periods of administration to increase the time for controlling weight loss and treating obesity.

### BACKGROUND OF THE DISCLOSURE

The present disclosure is generally directed to compositions and methods for controlling weight gain. Compositions for controlling weight include a combination of bupropion and a sympathomimetic drug. Methods for controlling weight and treating obesity include administering a composition having a combination of bupropion and a sympathomimetic drug.

Weight gain is an increase in body weight. Weight gain can be the result of an increase in muscle mass, fat deposits, or excess fluids. If enough weight is gained due to increased body fat deposits a person may become overweight in which the person has more body fat than is optimally healthy.

Uncontrolled weight gain can lead to obesity. Obesity is a medical condition in which excess body fat has accumulated to the extent that it may have an adverse effect on health. Obesity and overweightness are public health concerns. Obesity can result in an increased health problems and a shortened life expectancy. Obesity can also increase the likelihood of developing diseases such as, for example, heart disease, type 2 diabetes, obstructive sleep apnea, certain types of cancer, osteoarthritis and asthma.

Sympathomimetic drugs mimic the physiological effects of transmitter substances of the sympathetic nervous system. Sympathomimetic drugs can directly activate postsynaptic receptors and stimulate the production and release of catecholamines

Maintaining a healthy weight can control cholesterol, blood pressure and blood sugar, and can help to avoid or prevent diseases. Accordingly, there is a need for alternative compositions and methods for controlling weight gain and maintaining a healthy weight.

### SUMMARY OF THE DISCLOSURE

The present disclosure is generally directed to compositions and methods for treating weight gain. More specifically, in one aspect, the present disclosure is directed to a composition for treating weight gain comprising a combination of bupropion and a sympathomimetic drug.

In another aspect, the present disclosure is directed to a method for maintaining weight in an individual in need. The method comprises administering a composition comprising a combination of bupropion and a sympathomimetic drug.

In another aspect, the present disclosure is directed to a method of treating obesity in an individual in need. The method comprises administering a composition comprising a composition comprising a combination of bupropion and a sympathomimetic drug.

### DETAILED DESCRIPTION

While the disclosure is susceptible to various modifications and alternative forms, specific embodiments thereof are herein described below in detail. It should be understood, however, that the description of specific embodiments is not intended to limit the disclosure to cover all modifications, equivalents and alternatives falling within the spirit and scope of the disclosure as defined by the appended claims.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, suitable methods and materials are described below.

### Compositions

The present disclosure is generally directed to a composition for treating weight gain comprising a combination of bupropion and a sympathomimetic drug. The composition including a combination of bupropion and a sympathomimetic drug is useful for controlling weight of an individual in need and for treating obesity in an individual in need. A reduction of adverse drug reaction and potentiation of the anti-obesity activity are intended by the combination of bupropion and sympathomimetic drugs that have different mechanisms of action.

Without being bound by theory, bupropion ((±)-2-(tert-Butylamino)-1-(3-chlorophenyl)propan-1-one), shown as formula I, is known by those skilled in the art as an antidepressant that functions as a mild dopamine reuptake inhibitor and weak norepinephrine reuptake inhibitor. Bupropion is also known to function as a nicotinic acetylcholine receptor antagonist.

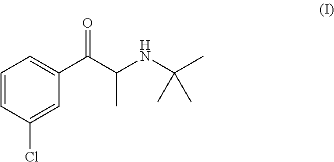 [SMILES: CC(NC(C)(C)C)C(=O)c1cccc(Cl)c1]

Sympathomimetic drugs mimic the physiological effects of transmitter substances of the sympathetic nervous system. Suitable sympathomimetic drugs can be, for example, dextroamphetamine ((2S)-1-phenylpropan-2-amine, d-alpha-methylphenethylamine dexamphetamine, dexamfetamine and DEXEDRINE®), ephedrine ((R*,S*)-2-(methylamino)-1-phenylpropan-1-ol), pseudoephedrine ((S, S)-2-methylamino-1-phenylpropan-1-ol), amphetamine ((RS)-1-phenylpropan-2-amine and (RS)-1-phenyl-2-aminopropane), methamphetamine (N-methyl-1-phenylpropan-2-amine), methylphenidate (methyl phenyl(piperidin-2-yl)acetate, CONCERTA® and RITALIN®), lisdexamfetamine ((2S)-2,6-diamino-N-[(2S)-1-phenylpropan-2-yl]hexanamide and VYVANSE®), cathinone ((S)-2-amino-1-phenyl-1-propanone), cathine ((1S,2S)-2-amino-1-phenylpropan-1-ol), methcathinone ((RS)-2-(methylamino)-1-phenyl-propan-1-one), benzylpiperazine (1-benzylpiperazine), methylenedioxypyrovalerone ((RS)-1-(Benzo[d] [1,3] dioxo1-5-yl)-2-(pyrrolidin-1-yl)pentan-1-one), 4-methylaminorex (4-Methyl-5 -phenyl-2-amino-oxazoline), pemoline ((RS)-2-amino-5-phenyl-1,3-oxazol-4(5H)-one and CYLERT®), phenmetrazine (3-methyl-2-phenylmorpholine and PRELUDIN®), phentermine, propylhexedrine ((RS)-N,α-dimethyl-cyclohexylethylamine, BENZEDREX® and OBESIN®) and combinations thereof

A particularly suitable sympathomimetic drug is phentermine (2-methyl-1-phenylpropan-2-amine) Without being bound by theory, phentermine, (shown as formula II) works on the hypothalamus portion of the brain to stimulate the adrenal glands to release norepinephrine, which can reduce hunger. Phentermine can also stimulate the release of epinephrine or adrenaline to reduce hunger and may cause fat cells to break down stored fat.

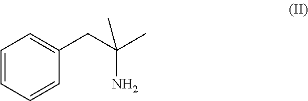

Phentermine is generally recommended as an appetite-suppressant for short-term treatment of obesity because tolerance can develop and lead to weight gain. The U.S. Food and Drug Administration recommend that phentermine be used for up to 12 weeks.

Another particularly suitable sympathomimetic drug is methylphenidate (CONCERTA® and RITALIN®) that can increase norepinephrine, epinephrine and dopamine in the brain.

In another embodiment, the composition can further include atomoxetine ((3R)-N-methyl-3 -(2-methylphenoxy)-3 -phenylpropan-1-amine; (R)-N-methyl-3 -phenyl-3 -(o-tolyloxy)propan-1-amine; STRATTERA®). Atomoxetine is approved by the FDA for the treatment of ADHD and selectively inhibits norepinephrine reuptake.

In one embodiment, the composition for treating weight gain includes a combination of a therapeutically effective amount of bupropion and a therapeutically effective amount of a sympathomimetic drug. As used herein, “therapeutically effective amount” refers to that amount that provides a therapeutic effect for a given condition and administration regimen. It can be understood, however, that the total daily usage of the compounds and compositions of the disclosure can be decided by the attending physician within the scope of sound medical judgment. The specific effective dose level for any particular patient can depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts.

Actual dosage levels of active ingredients in the compositions disclosed herein can be varied so as to obtain an amount of the active compound(s) that is effective to achieve the desired response for a particular individual, composition and mode of administration. The selected dosage level can depend upon the activity of the particular compound, the route of administration, the severity of the condition being treated and the condition and prior medical history of the individual being treated. However, it is within the skill of the art to start doses of the composition at levels lower than required to achieve the desired effect and to gradually increase the dosage until the desired effect is achieved. Upon achieving the desired effect, a fixed ratio of each agent can be determined to form a concentrated dosage of the composition to accommodate a pill, capsule or tablet such that the composition can be administered over a period of time such as, for example, a 24 hour period.

The active compounds (e.g., bupropion and sympathomimetic drugs) can be administered in a convenient manner such as by oral administration, injection (subcutaneous, intravenous, etc.), inhalation, transdermal application, or rectal administration. A preferred route of administration is by oral administration. Depending on the route of administration, the active compound may be coated in a material to protect the compound from the action of enzymes, acids and other natural conditions which may inactivate the compound. Preparations of the active compounds can further be in the form of pharmaceutically acceptable salts and pharmaceutically acceptable solvates. Preparations of the compositions can further be in time-release forms such as, for example, instant-release, sustained-release, controlled-release, slow-release, modified-release, and extended-release. As known by those skilled in the art, time-release technology refers to a mechanism to dissolve a drug over time in order to be released slower and steadier into the bloodstream while having the advantage of being taken at less frequent intervals than immediate-release formulations of the same drug. Time-release formulations can be prepared by embedding one or both of the active compounds in a matrix of insoluble substances such that the dissolving v must find its way out through the holes in the matrix. Other time-release formulations can be prepared by enclosing active compounds in polymer-based tablets whereby stomach acids push the active compounds out. Other time-release formulations can be prepared by embedding the active compounds in a matrix that swells into a gel thereby allowing the active compounds to exit through the outer surface of the gel. Other time-release formulations can be prepared by microencapsulation whereby the active compounds coated around an inert core are layering with insoluble substances to form a microsphere.

As used herein, “dosage” refers to physically discrete units suited as unitary dosages for the individual to be treated; each unit containing a predetermined quantity of active compound calculated to produce the desired effect in association with a pharmaceutical carrier. The specification for the dosage unit forms of the present disclosure are dictated by and directly dependent on (a) the unique characteristics of the active compound and the particular effect to be achieved, and (b) the limitations inherent in the art of compounding such an active compound for the treatment of an individual.

The composition can also include a pharmaceutically acceptable carrier. The term “pharmaceutically acceptable carrier” refers to any suitable adjuvant, carrier, excipient, additive, antioxidant, osmolality adjusting agent, buffer, pH adjusting agent, or stabilizer. The pharmaceutically acceptable carrier can be in solid or liquid form such as, pills, tablets, capsules, powders, solutions, suspensions, or emulsions. Particularly suitable carriers can be pill-form such as, for example, a tablet and a capsule. Suitable pharmaceutically carriers can be, for example, saline (solution of 0.90% w/v of NaC1), half-normal saline (solution of 0.45% NaCl), quarter-normal saline (solution of 0.22% NaCl), D5W (a 5% dextrose in water solution), DSNS (a 5% dextrose in normal saline solution), Ringer's lactate, and other solutions known by those skilled in the art.

A particularly suitable composition for oral administration can be a pill-form (for example, a tablet and a capsule) containing bupropion and phentermine The dosage of bupropion can be, for example, from about 75 mg to about 400 mg, including from about 75 mg to about 100 mg, from about 75 mg to about 150 mg, from about 75 mg to about 200 mg, and from about 75 mg to about 300 mg. The dosage of phentermine can be, for example, from about 8 mg to about 37.5 mg, including from about 8 mg to about 10 mg, from about 8 mg to about 15 mg, and from about 8 mg to about 30 mg. In one particularly suitable embodiment, the composition can include about 400 mg of bupropion and from about 8 mg phentermine to about 37.5 mg phentermine. In another particularly suitable embodiment, the composition can include about 400 mg of bupropion and about 8 mg phentermine In another particularly suitable embodiment, the composition can include about 400 mg of bupropion and about 10 mg phentermine In another particularly suitable embodiment, the composition can include about 400 mg of bupropion and about 15 mg phentermine. In another particularly suitable embodiment, the composition can include about 400 mg of bupropion and about 30 mg phentermine In another particularly suitable embodiment, the composition can include about 400 mg of bupropion and about 37.5 mg phentermine For adults, the dosages of methylphenidate (RITALIN®) can be from about 10 mg/day to about 60 mg/day and can be administered in two to three divided doses per day. For adolescents age 13 to 17 years of age, the dosages of methylphenidate (CONCERTA®) can be from about 18 mg/day to about 72 mg/day. For adults 18 years and older, the dosages of methylphenidate (CONCERTA®) can be from about 18 mg/day to about 72 mg/day. For adults, the dosage of atomoxetine can be from about 20 mg/day to about 100 mg/day and can be administered in 2 divided doses per day.

For children age 6 years and older, the dosage of bupropion can be from about 1 mg/kg of body weight per day to about 6 mg/kg of body weight per day. For children age 6 and up with a body weight up to 70 kg, the dosages of atomoxetine can be from about 0.5 mg/kg of body weight per day to about 1.4 mg/kg of body weight per day. For children age 6 years and older with a body weight over 70 kg, the dosages of atomoxetine can be from about 20 mg/day to about 40 mg/day. For children age 6 years and older, the dosages of methylphenidate (RITALIN®) can be from about 0.3 mg/kg of body weight per day to about 2 mg/kg of body weight per day in three (3) divided doses or from about 5 mg twice per day to about 30 mg twice per day up to 60 mg per day. For children age 6 to 12 years of age, the dosages of methylphenidate (CONCERTA®) can be from about 18 mg/day to about 54 mg/day.

### Methods for Controlling Weight

In another aspect, the present disclosure is directed to a method for controlling weight in an individual in need. The method includes administering to in an individual in need a composition including a combination of bupropion and a sympathomimetic drug.

Bupropion as a single agent is effective for treating obesity at a dose of 400 mg per day.

Sympathomimetic drugs mimic the physiological effects of transmitter substances of the sympathetic nervous system. Suitable sympathomimetic drugs can be, for example, dextroamphetamine ((2S)-1-phenylpropan-2-amine, d-alpha-methylphenethylamine; dexamphetamine, dexamfetamine and DEXEDRINEO), ephedrine ((R*,S*)-2-(methylamino)-1-phenylpropan-1-ol), pseudoephedrine ((S,S)-2-methylamino-1-phenylpropan-1-ol), amphetamine ((RS)-1-phenylpropan-2-amine and (RS)-1-phenyl-2-aminopropane), methamphetamine (N-methyl-1-phenylpropan-2-amine), methyl phenidate (methyl phenyl(piperidin-2-yl)acetate, CONCERTA® and RITALIN®), lisdexamfetamine ((25)-2,6-diamino-N-[(2S)-1-phenylpropan-2-yl]hexanamide and VYVANSE®), cathinone ((S)-2-amino-1-phenyl-1-propanone), cathine ((1S,2 S)-2-amino-1-phenylpropan-1-ol), methcathinone ((RS)-2-(methylamino)-1 -phenyl-propan-1-one), benzylpiperazine (1-benzylpiperazine), methylenedioxypyrovalerone ((RS)-1-(Benzo [d] [1,3 ]dioxo1-5 -yl)-2-(pyrrolidin-1-yl)pentan-1-one), 4-methylaminorex (4-Methyl-5-phenyl-2-amino-oxazoline), pemoline ((RS)-2-amino-5-phenyl-1,3-oxazol-4(5H)-one and CYLERT®), phenmetrazine (3-methyl-2-phenylmorpholine and PRELUDIN®), phentermine and propylhexedrine ((RS)-N,α-dimethyl-cyclohexylethylamine and BENZEDREX® and OBESIN®) and combinations thereof

A particularly suitable sympathomimetic drug is phentermine Phentermine is generally recommended as an appetite-suppressant for short-term treatment for obesity because tolerance can develop and lead to weight gain. The U.S. Food and Drug Administration recommend that phentermine be used for up to 12 weeks. The U.S. Food and Drug Administration also recommends a dosage of a half of a single 37.5 mg phentermine hydrochloride tablet up to a single 37.5 mg phentermine hydrochloride tablet (equivalent to 30 mg phentermine base) for patients with a body mass index of greater than 30 kg/m² (or greater than 27 kg/m² with other risk factors such as controlled hypertension, diabetes, and hyperlipidemia).

In using the composition of the present disclosure, the same dose of bupropion and of sympathomimetic drug that is usually given as a single agent may be given through a normal administration route. In a particularly preferred embodiment in using the composition having a combination of bupropion and phentermine, a reduced dose (for example, 0.10-0.99 times the highest dose as a single agent) of bupropion and sympathomimetic drug can be given through a normal administration route. Particularly suitable dosages for adults, adolescent children and children are described herein.

The individual in need can be overweight and/or at risk for becoming overweight. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein.

As used herein, “controlling weight” refers to maintaining the individual's body weight such that the individual's body weight remains unchanged, is sustained and/or fluctuates within an acceptable range as determined by those skilled in the art such as, for example, medical professionals, dietitians, nutritionists and other health professionals. As used herein, “controlling weight” also refers managing an individual's weight to avoid or reduce weight gain or to stimulate or result in weight loss.

The effect of administering the composition including a combination of bupropion and a sympathomimetic drug can be confirmed by a reduction in the individual's body weight in comparison with that of the individual's body weight prior to administration. The effect of administering the composition including a combination of bupropion and a sympathomimetic drug can also be confirmed by other factors such as, for example, a reduction in the individual's body mass index, reduction in waist circumference, improvement in waist-hip ratio, reduction of body fat (either or both of percentage body fat and total body fat) and combinations thereof A particularly suitable effect of administering the composition including a combination of bupropion and a sympathomimetic drug for controlling weight would be to maintain an individual's body mass index to less than about 30 kg/m². Another particularly suitable effect of administering the composition including a combination of bupropion and a sympathomimetic drug for controlling weight would be to maintain an individual's body mass index to a range of from about 25 kg/m² to about 29.9 kg/m². An even more suitable effect of administering the composition including a combination of bupropion and a sympathomimetic drug for controlling weight would be to maintain an individual's body mass index to a range of from about 18.5 kg/m² to about 24.9 kg/m².

As used herein, the term “individual” refers to the term “individual” refers to a male human or a female human. The individual can be, for example, a human. The individual can be an adult individual, an adolescent child (age 6 to 18 years) and a child (less than 6 years).

The composition can also include a pharmaceutically acceptable carrier as described herein for administration. Particularly suitable carriers can be pill-form such as, for example, a tablet and a capsule.

The composition can be administered in any manner effective for delivering the compositions. Exemplary modes of administration may be, for example, orally, topically, transdermally, parenterally, enterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes. A particularly suitable administration can be oral administration.

The compounds described herein are administered as a combination. It will be appreciated that when using a combination the agents may be in the same pharmaceutically acceptable carrier at a specific ratio that is administered simultaneously in the same pharmaceutically acceptable carrier to achieve the desired effect. Buproprion and its metabolites such as, for example, R,R-hydroxybupropion, S,S-hydroxybupropion, threo-hydrobupropion and erythro-hydrobupropion, can be monitored for example, by measurement in the blood and using positron emission tomography of the brain. Similarly, phentermine and its metabolites can be measured in the blood. The ratio of each agent can be determined and/or adjusted by one skilled in the art using methods known by those skilled in the art such as, for example, monitoring bupropion and its metabolites using for example, positron emission tomography of the brain, measuring bupropion and its metabolites in the blood and other methods. Thus, the individual receives the composition containing the specific ratio of each compound to achieve the desired effect. The composition can also be in separate pharmaceutical carriers such as conventional dosage forms that are administered simultaneously such that each compound is administered at the specific ratio. For example, the compounds forming the composition can be in separate pill forms that are co-administered to the individual. Administration of each compound may be effected by any appropriate route such as, for example, oral routes, intravenous routes, intramuscular routes, and direct absorption through mucous membrane tissues. The compounds may be administered by the same route or by different routes. For example, a first compound of the composition may be administered orally while the other compound of the composition may be administered by a non-oral route. The amounts of each compound are administered at a dosage of each to arrive at the specific ratio desired to achieve the desired effect.

A suitable method for oral administration can be a pill-form (for example, a capsule and a tablet) containing bupropion and a sympathomimetic drug. A particularly suitable form for oral administration can be a pill-form (for example, a tablet and a capsule) containing bupropion and phentermine The dosage of bupropion can be, for example, from about 75 mg to about 400 mg, including from about 75 mg to about 100 mg, from about 75 mg to about 150 mg, from about 75 mg to about 200 mg, and from about 75 mg to about 300 mg. The dosage of phentermine can be, for example, from about 8 mg to about 37.5 mg, including from about 8 mg to about 10 mg, from about 8 mg to about 15 mg, and from about 8 mg to about 30 mg. In one particularly suitable embodiment, the combination therapy can include about 400 mg of bupropion and from about 8 mg phentermine to about 37.5 mg phentermine In another particularly suitable embodiment, the composition can include about 400 mg of bupropion and about 8 mg phentermine In another particularly suitable embodiment, the composition can include about 400 mg of bupropion and about 10 mg phentermine In another particularly suitable embodiment, the combination therapy can include about 400 mg of bupropion and about 15 mg phentermine. In another particularly suitable embodiment, the composition can include about 400 mg of bupropion and about 30 mg phentermine In another particularly suitable embodiment, the composition can include about 400 mg of bupropion and about 37.5 mg phentermine. For adults, the dosages of methylphenidate (RITALIN®) can be from about 10 to about 60 mg per day and can be administered in two to three divided doses per day. For adolescents age 13 to 17 years of age, the dosages of methylphenidate (CONCERTA®) can be from about 18 mg/day to about 72 mg/day. For adults 18 years and older, the dosages of methylphenidate (CONCERTA®) can be from about 18 mg/day to about 72 mg/day. For adults, the dosage of atomoxetine can be from about 20 mg per day to about 100 mg per day and can be administered in 2 divided doses per day.

For children age 6 years and older, the dosage of bupropion can be from about 1 mg/kg of body weight per day to about 6 mg/kg of body weight per day. For children age 6 and up with a body weight up to 70 kg, the dosages of atomoxetine can be from about 0.5 mg/kg of body weight per day to about 1.4 mg/kg of body weight per day. For children age 6 years and older with a body weight over 70 kg, the dosages of atomoxetine can be from about 20 mg per day to about 40 mg per day. For children age 6 years and older, the dosages of methylphenidate (RITALIN®) can be from about 0.3 mg/kg of body weight per day to about 2 mg/kg of body weight per day in three (3) divided doses or from about 5 mg twice per day to about 30 mg twice per day up to 60 mg per day. For children age 6 to 12 years of age, the dosages of methylphenidate (CONCERTA®) can be from about 18 mg/day to about 54 mg/day.

### Methods for Treating Obesity

In another aspect, the present disclosure is directed to a method for treating obesity in an individual in need. The method includes administering to in an individual in need a composition including a combination of bupropion and a sympathomimetic drug.

Suitable sympathomimetic drugs for use in the method for treating obesity can be those described herein. A particularly suitable sympathomimetic drug for use in the method of treating obesity is phentermine, as described herein.

In another embodiment, the method can further include administering atomoxetine, as described herein.

In using the composition of the present disclosure, the same dose of bupropion and of phentermine that is usually given as a single agent may be given through a normal administration route. In a particularly preferred embodiment in using the combination therapy of bupropion and phentermine, a reduced dose (for example, 0.10-0.99 times the highest dose as a single agent) of bupropion and the sympathomimetic drug can be given through a normal administration route. A particularly suitable form for oral administration can be a pill-form (for example, a tablet and a capsule) containing bupropion and phentermine.

The individual in need can be obese and/or at risk for obesity. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein.

Obesity and whether an individual is at risk for obesity can be determined by one skilled in the art such as, for example, medical professionals, dietitians, nutritionists and other health professionals. To determine whether an individual is obese or at risk for obesity, body mass index can be calculated, waist circumference can be determined, waist-hip ratio can be determined, body fat (either or both of percentage body fat and total body fat) can be determined, family history can be considered, genetic susceptibility can be considered, sedentary lifestyle of the individual, whether the individual suffers from or is at risk of having an endocrine disorder and combinations thereof. As established by the World Health Organization, an individual is considered to be obese when their body mass index exceeds 30 kg/m². An individual is considered to be overweight at a body mass index of from about 25 kg/m² to about 29.9 kg/m². An individual is considered to be of normal weight at a body mass index of from about 18.5 kg/m²to about 24.9 kg/m².

The effect of administering the composition including a combination of bupropion and a sympathomimetic drug can be confirmed by a reduction in the individual's body weight in comparison with that of the individual's body weight prior to administration. The effect of administering the composition including a combination of bupropion and a sympathomimetic drug can also be confirmed by other factors such as, for example, a reduction in the individual's body mass index, reduction in waist circumference, improvement in waist-hip ratio, reduction of body fat (either or both of percentage body fat and total body fat) and combinations thereof A particularly suitable effect of administering the composition including a combination of bupropion and a sympathomimetic drug would be a reduction in body mass index to less than about 30 kg/m². Another particularly suitable effect of administering the composition including a combination of bupropion and a sympathomimetic drug would be a reduction in body mass index to a range of from about 25 kg/m² to about 29.9 kg/m². An even more suitable effect of administering the composition including a combination of bupropion and a sympathomimetic drug would be a reduction in body mass index to a range of from about 18.5 kg/m² to about 24.9 kg/m².

As used herein, the term “individual” refers to any mammalian subject. The individual can be, for example, a human. The individual can be, for example, a human. The individual can be an adult individual, a child, and a pediatric individual.

The composition including a combination of bupropion and a sympathomimetic drug can also include a pharmaceutically acceptable carrier as described herein for administration. Particularly suitable carriers can be pill-form such as, for example, a tablet and a capsule.

The composition can be administered in any manner effective for delivering the composition. Exemplary modes of administration may be, for example, orally, topically, transdermally, parenterally, enterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. A particularly suitable administration can be oral administration.

The agents of the composition described herein are administered as a combination. It will be appreciated that when using a combination the agents may be in the same pharmaceutically acceptable carrier at a specific ratio that is administered simultaneously in the same pharmaceutically acceptable carrier to achieve the desired effect, as described herein. The agents of the composition can also be in separate pharmaceutical carriers such as conventional dosage forms that are administered simultaneously such that each agent is administered at the specific ratio, as described herein.

A particularly suitable composition for oral administration can be a pill-form (for example, a tablet and a capsule) containing bupropion and phentermine. The dosage of bupropion can be, for example, from about 75 mg to about 400 mg, including from about 75 mg to about 100 mg, from about 75 mg to about 150 mg, from about 75 mg to about 200 mg, and from about 75 mg to about 300 mg. The dosage of phentermine can be, for example, from about 8 mg to about 37.5 mg, including from about 8 mg to about 10 mg, from about 8 mg to about 15 mg, and from about 8 mg to about 30 mg. In one particularly suitable embodiment, the composition can include about 400 mg of bupropion and from about 8 mg phentermine to about 37.5 mg phentermine. In another particularly suitable embodiment, the composition can include about 400 mg of bupropion and about 8 mg phentermine In another particularly suitable embodiment, the composition can include about 400 mg of bupropion and about 10 mg phentermine In another particularly suitable embodiment, the composition can include about 400 mg of bupropion and about 15 mg phentermine. In another particularly suitable embodiment, the composition can include about 400 mg of bupropion and about 30 mg phentermine In another particularly suitable embodiment, the composition can include about 400 mg of bupropion and about 37.5 mg phentermine For adults, the dosages of methylphenidate (RITALIN®) can be from about 10 to about 60 mg per day and can be administered in two to three divided doses per day. For adolescents age 13 to 17 years of age, the dosages of methylphenidate (CONCERTA®) can be from about 18 mg/day to about 72 mg/day. For adults 18 years and older, the dosages of methylphenidate (CONCERTA®) can be from about 18 mg/day to about 72 mg/day.

For adults, the dosage of atomoxetine can be from about 20 mg per day to about 100 mg per day and can be administered in 2 divided doses per day.

For children age 6 years and older, the dosage of bupropion can be from about 1 mg/kg of body weight per day to about 6 mg/kg of body weight per day. For children age 6 and up with a body weight up to 70 kg, the dosages of atomoxetine can be from about 0.5 mg/kg of body weight per day to about 1.4 mg/kg of body weight per day. For children age 6 years and older with a body weight over 70 kg, the dosages of atomoxetine can be from about 20 mg per day to about 40 mg per day. For children age 6 years and older, the dosages of methylphenidate (RITALIN®) can be from about 0.3 mg/kg of body weight per day to about 2 mg/kg of body weight per day in three (3) divided doses or from about 5 mg twice per day to about 30 mg twice per day up to 60 mg per day. For children age 6 to 12 years of age, the dosages of methylphenidate (CONCERTA®) can be from about 18 mg/day to about 54 mg/day.

### EXAMPLES

### Example 1

In this Example, a composition including bupropion and phentermine was administered to an individual for treating weight gain.

A combination of 200 mg of bupropion and 18.75 mg of phentermine was administered to a male individual over a 3 week period. Starting weight was 205 lbs and ending weight was 195 lbs.

What is claimed is:

1. A composition for controlling weight comprising: bupropion and a sympathomimetic drug.

2. The composition of claim 1, wherein the bupropion comprises from about 75 mg to about 400 mg.

3. The composition of claim 1, wherein the sympathomimetic drug is selected from the group consisting of dextroamphetamine, ephedrine, pseudoephedrine, amphetamine, methamphetamine, methyl phenidate, lisdexamfetamine, cathinone, cathine, methcathinone, benzylpiperazine, methylenedioxypyrovalerone, 4-methylaminorex, pemoline, phenmetrazine, phentermine and propylhexedrine.

4. The composition of claim 1, wherein the sympathomimetic drug is phentermine.

5. The composition of claim 4, wherein the phentermine comprises from about 8 mg to about 37.5 mg.

6. A method for controlling weight of an individual in need, the method comprising: administering to the individual a composition comprising bupropion and a sympathomimetic drug.

7. The method of claim 6, wherein the bupropion comprises from about 75 mg to about 400 mg.

8. The method of claim 6, wherein the sympathomimetic drug is selected from the group consisting of dextroamphetamine, ephedrine, pseudoephedrine, amphetamine, methamphetamine, methyl phenidate, lisdexamfetamine, cathinone, cathine, methcathinone, benzylpiperazine, methylenedioxypyrovalerone, 4-methylaminorex, pemoline, phenmetrazine, phentermine, propylhexedrine, and combinations thereof.

9. The method of claim 8, wherein the sympathomimetic drug is phentermine.

10. The method of claim 9, wherein the phentermine comprises from about 8 mg to about 37.5 mg.

11. The method of claim 6, wherein the individual's body mass index is maintained at a range of from about 25 kg/m² to about 29.9 kg/m².

12. The method of claim 6, wherein the individual's body mass index is maintained at a range of from about 18.5 kg/m² to about 24.9 kg/m².

13. The method of claim 6, wherein the administering is selected from the group consisting of orally, topically, transdermally, parenterally, subcutaneously, intravenously, intramuscularly, intraperitoneally, by intranasal instillation, by intracavitary or intravesical instillation, intraocularly, intraarterially, intralesionally, and by application to mucous membranes.

14. A method for treating obesity in an individual in need, the method comprising: administering to the individual composition comprising bupropion and a sympathomimetic drug.

15. The method of claim 14, wherein the bupropion comprises from about 75 mg to about 400 mg.

16. The method of claim 14, wherein the sympathomimetic drug is selected from the group consisting of dextroamphetamine, ephedrine, pseudoephedrine, amphetamine, methamphetamine, methylphenidate, lisdexamfetamine, cathinone, cathine, methcathinone, benzylpiperazine, methylenedioxypyrovalerone, 4-methylaminorex, pemoline, phenmetrazine, phentermine, propylhexedrine and combinations thereof.

17. The method of claim 16, wherein the sympathomimetic drug is phentermine.

18. The method of claim 17, wherein the phentermine comprises from about 8 mg to about 37.5 mg.

19. The method of claim 14, wherein the individual's body mass index is maintained at a range of from about 25 kg/m² to about 29.9 kg/m².

20. The method of claim 14, wherein the individual's body mass index is maintained at a range of from about 18.5 kg/m² to about 24.9 kg/m².
